(19) United States
(12) Patent Application Publication
(10) Pub. No.: US 2005/0260207 A1
(43) Pub. Date: Nov. 24, 2005

(54) Monoclonal antibody neutralising cathepsin b activity and uses thereof

(76) Inventors: Janko Kos, Domzale (SI); Ales Premzl, Maribor (SI); Natasa Kopitar Jerala, Ljubljana (SI); Xiaohui Fan, Ljubljana (SI); Vito Turk, Ljubljana (SI); Marco Bestagno, Trieste (IT); Oscar R Burrone, Trieste (IT)

Correspondence Address: Burto A Amernick;Connolly Bove Lodge & Hutz, PO Box 19088, Washington, DC, 20036-0088, US

(21) Appl. No.: 10/477,950

(22) PCT Filed: Apr. 2, 2002
(86) PCT No.: PCT/SI02/00013

§ 371 Date: May 24, 2004

(30) Foreign Application Priority Data
May 18, 2001 (SI) P 200100132

(51) Int. Cl. A61K 39/395

(52) U.S. Cl. 424/155.1

(57) ABSTRACT

The present invention relates to a monoclonal antibody capable of neutralising cathepsin B. In particular, the present invention is concerned with the use of such an antibody for the detection or treatment of diseases associated with an over-expression and/or excessive activity of cathepsin B, such as cancer or arthritis.

### TECHNICAL FIELD OF INVENTION

The present invention relates to a monoclonal antibody, capable of neutralising cathepsin B activity. In particular, the present invention is concerned with the use of such an antibody for the treatment and detection of diseases associated with an over-expression and/or excessive activity of cathepsin B, such as cancer or arthritis.

### BACKGROUND OF INVENTION

Lysosomal cysteine proteinase cathepsin B (Cat B) has been shown to participate in processes of tumour growth, invasion and metastasis (Kos, J. and Lah, T. T., Oncology Reports 5: 1349-1361, 1996). It has been shown that tumour cathepsin B can be translocated to the plasma membrane or secreted either as a pro-form or as an active enzyme from tumour cells where it seems to take part in the degradation of the components of extracellular matrix and basement membrane, which is deemed a crucial step in the metastatic process (Sloane et al., Biochemical and Molecular Aspects of Selected Cancers, T. G. Pretlow and T. P. Pretlow eds., Academic Press, New York, pp. 411-465, 1994). Cathepsin B activity is typically controlled by endogenous inhibitors of cysteine proteinases—such as the intracellular stefins A and B and extracellular cystatins, kininogens and α2-macroglobulin. It has been shown that the increased level of tumour cathepsin B is not balanced by a corresponding increase of cysteine proteinase inhibitors, which may lead to an uncontrolled proteolysis of the extracellular matrix. In clinical studies of breast, head and neck, colorectal and lung cancers, increased Cat B activity in the tumour tissue and increased protein concentration correlated with more aggressive tumour behaviour, early relapse and shorter overall survival (Kos, J. and Lah, T. T., Oncology Reports 5: 1349-1361, 1996). Significantly increased levels of Cat B have also been found in sera of patients with breast, colorectal, liver, pancreatic and melanoma cancers (Kos et al., Int. J. Biol. Markers, 15:84-89, 2000).

On the other hand, a decrease in inhibitory ability was also proposed to account for an inadequate control of cathepsin B in cancer progression. For example, stefin A purified from human sarcoma exhibited a lower inhibitory activity as compared to liver stefin A (Lah et al., Biochim. Biophys. Acta 993: 63-73, 1989). In lung tumour tissue cathepsin B was more resistant to inactivation by E-64 than cathepsin B from control lung tissue (Krepela et al., Int. J. Cancer 61: 44-53, 1995). Additionally, cathepsin B from more metastatic lung cells exhibited different rates of inhibition by E-64 than the enzyme from less metastatic lung cancer cell lines (Spiess et al., J. Histochem. Cytochem. 42: 917-929, 1994). The level of cathepsin B/cystatin C complex was shown to be lower in sera of patients with lung and colorectal cancers compared to those with benign diseases or healthy controls (Zore et al., Biol. Chem. 382: 2001).

At present it seems that in cancer patients the ability of endogenous inhibitors of cysteine proteinases to effectively balance an over-expression and/or an excessive activity of tumour associated cysteine proteinases is compromised. Yet, there is no direct evidence for tumour associated factors affecting the inhibition of cathepsin B in vivo, however, there are several in vitro studies reporting tumour associated post-translational modifications of cathepsin B, changes in pH stability, the presence of activators or the binding of glycosaminoglycans (GAGs), which all may change the conformation of cathepsin B active site and the consequent binding of the inhibitors (Zore et al., Biol. Chem. 382: 2001).

It has also been reported (Kobayashi, H. et al. (1992) Cancer Research 52: 3610-3614) that membrane associated cathepsin B may play an indirect role in cancer invasion, activating pro-uPA. In this paper it was not demonstrated that the polyclonal antibody neutralises cathepsin B endopeptidase activity and, consequently, the invasion of tumour cells. A polyclonal antibody was used without proven inhibitory activity against cathepsin B.

Since cysteine proteinase inhibitors could provide a therapeutic tool for the treatment of cancer, various natural protein inhibitors as well as their synthetic analogues have been prepared and tested for anti-tumour effect. Unfortunately, the specificity of natural inhibitors is not limited to one particular enzyme. Further, small synthetic inhibitors, reversible and irreversible, proved to be cytotoxic at higher concentrations. Consequently, there exists a need for additional tools for treating cancer and other disorders associated with over-expression and/or excessive activity of cathepsin B such as arthritis, autoimmune diseases, asthma, neurodegenerative disorders, periodontal disease, muscular dystrophy, osteoporosis, etc.

### SUMMARY OF INVENTION

In the course of the extensive studies leading to the present invention, the inventors have found that neutralising monoclonal antibodies directed against cathepsin B provide an intriguing opportunity for specific inhibition of said proteolytic activity of said enzyme.

Consequently, according to a first aspect, the present invention provides for neutralising monoclonal antibodies directed against cathepsin B so as to impair its biological activity.

According to another embodiment, the invention provides a monoclonal antibody recognizing cathepsin B and impairing its biological activity, wherein the antibody comprises murine variable regions and human constant regions (chimeric antibody).

According to still another aspect, the present invention provides humanised monoclonal antibodies having the above traits.

The present invention also provides polypeptide fragments comprising only a portion of the primary antibody structure, which possess one or more immunoglobulin activities (mini-antibodies).

According to still another aspect, the present invention provides a hybridoma cell line expressing such a monoclonal antibody, which was deposited with Deutsche Sammlung von Mikroorganismen und Zellkulturen GmbH (DSMZ), Mascheroder Weg 1b, D-38124 Braunschweig, Germany on 17.5.2001 and received the accession No. DSM ACC2506. DSMZ has the status of International Depositary Authority according to Budapest Treaty.

The present invention also provides for the use of antibodies described herein for the treatment and/or diagnosis of diseases associated with over-expression of cathepsin B and/or its excessive activity. Such diseases are in particular cancer or arthritis.

### DETAILED DESCRIPTION OF THE INVENTION

In the Figs.,

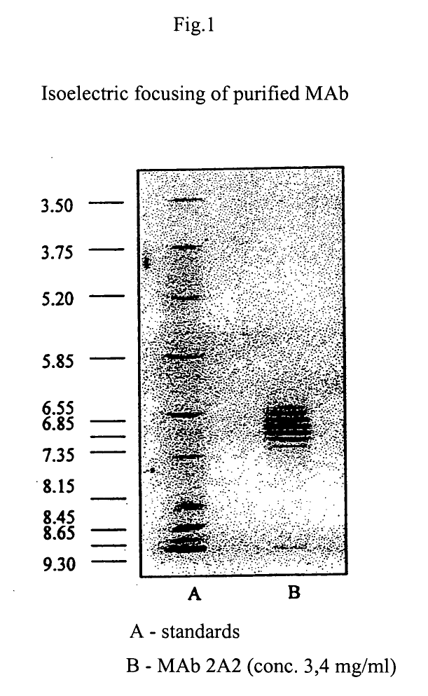

FIG. 1 shows the results of an isoelectric focusing of 2A2 monoclonal antibody. A set of bands was focused in a pI range between 6.55 and 7.2, showing the monoclonality of the antibody.

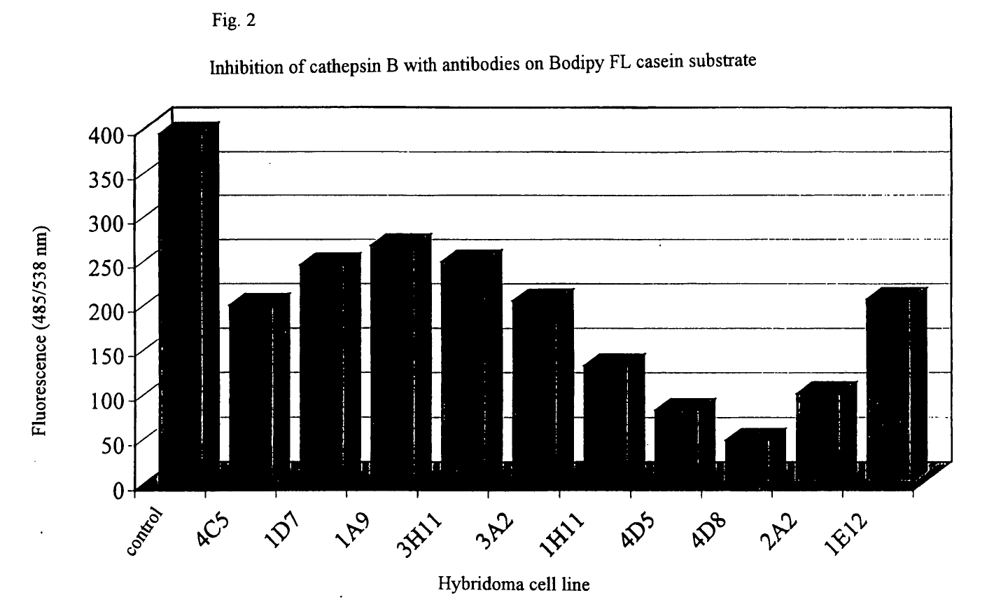

FIG. 2 shows the results of inhibition of cathepsin B activity on BODIPYL FL casein substrate using neutralising anti-cathepsin B antibodies. Cathepsin B was used as a positive control.

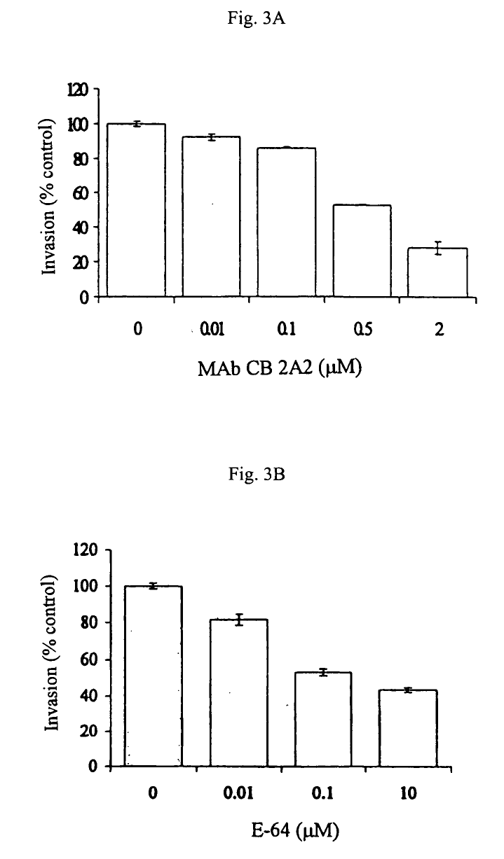

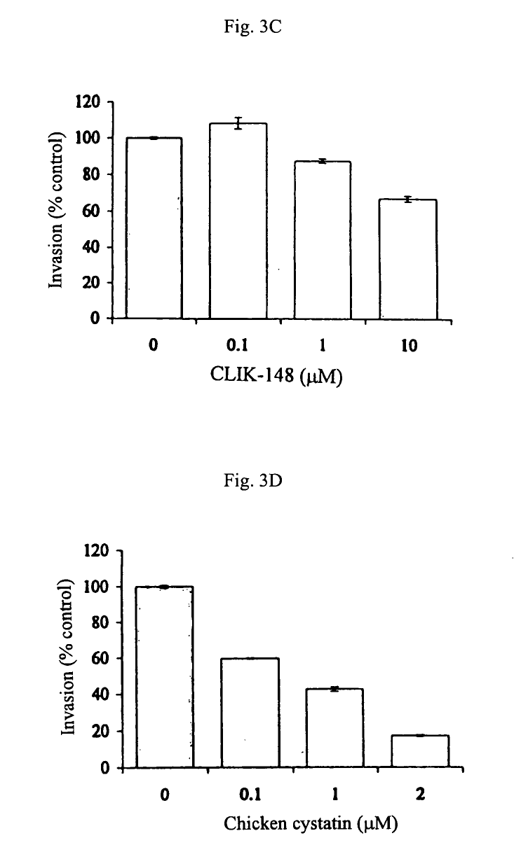

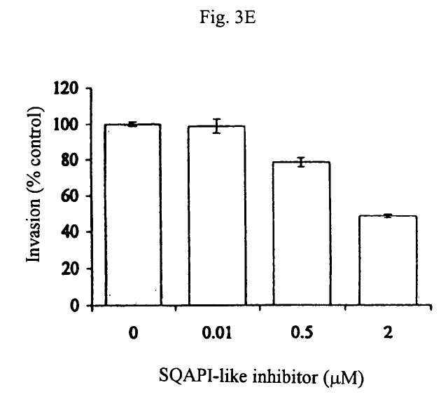

FIGS. 3A-3E show the results of inhibition of invasion of MCF-10A neoT cells through Matrigel by monoclonal antibody (MAb) according to the invention (FIG. 3A) and, for comparison, the results of inhibition by irreversible inhibitor E-64 (FIG. 3B), CLIK-148 (FIG. 3C), chicken cystatin (FIG. 3D) and a SQAPI-like inhibitor (FIG. 3E). To define the molar concentration of 2A2 monoclonal antibody, it was considered as bivalent inhibitor.

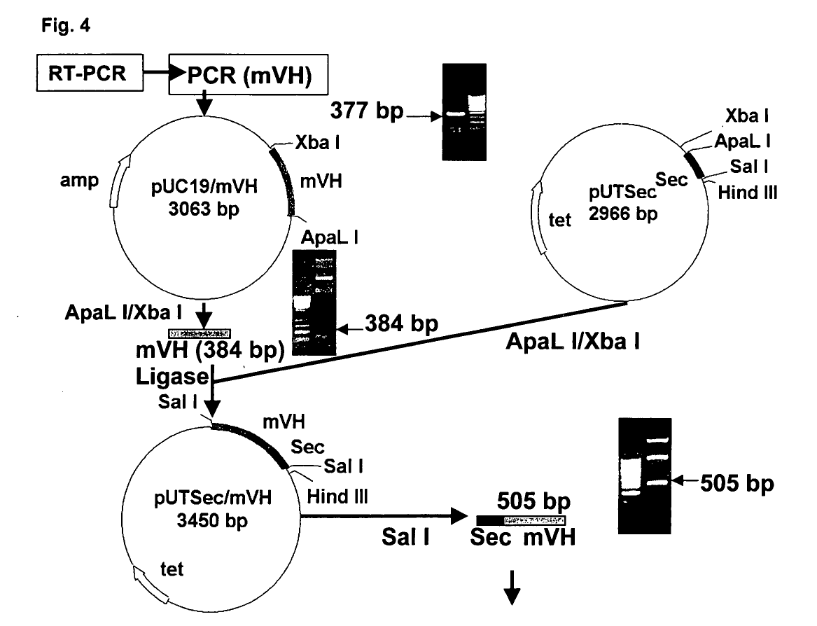

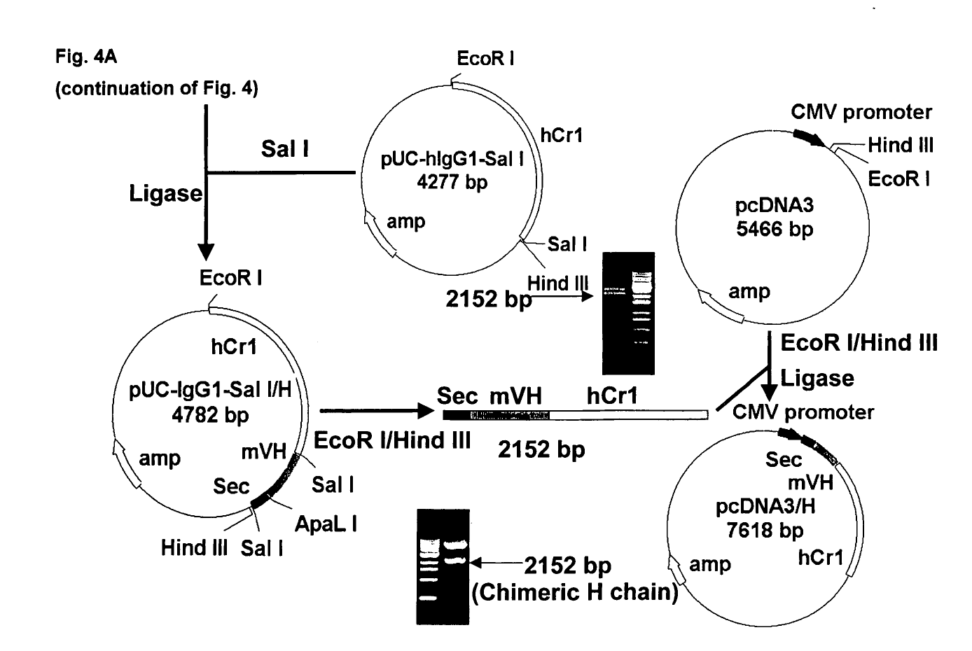

FIGS. 4 and 4A show a scheme for the construction of a chimeric heavy chain.

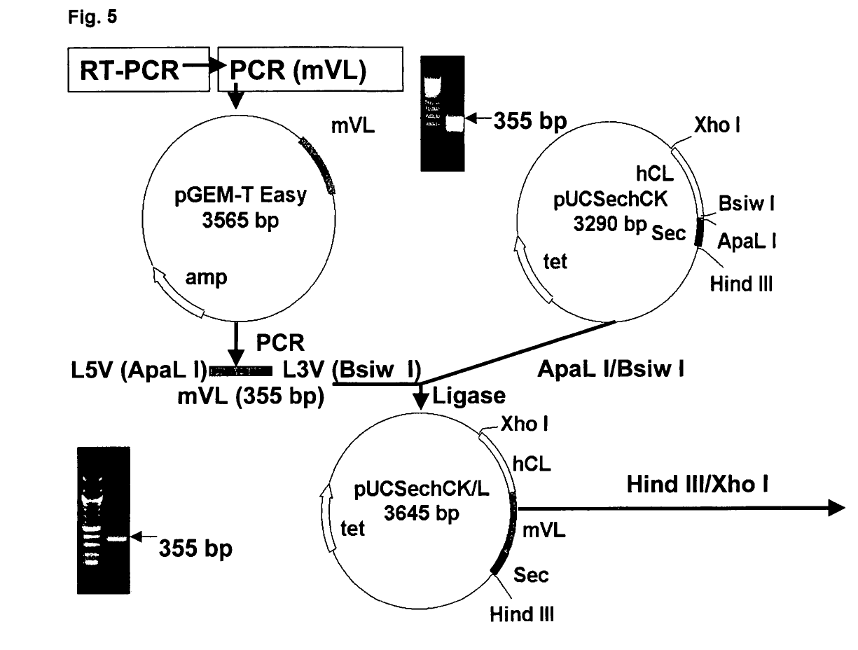

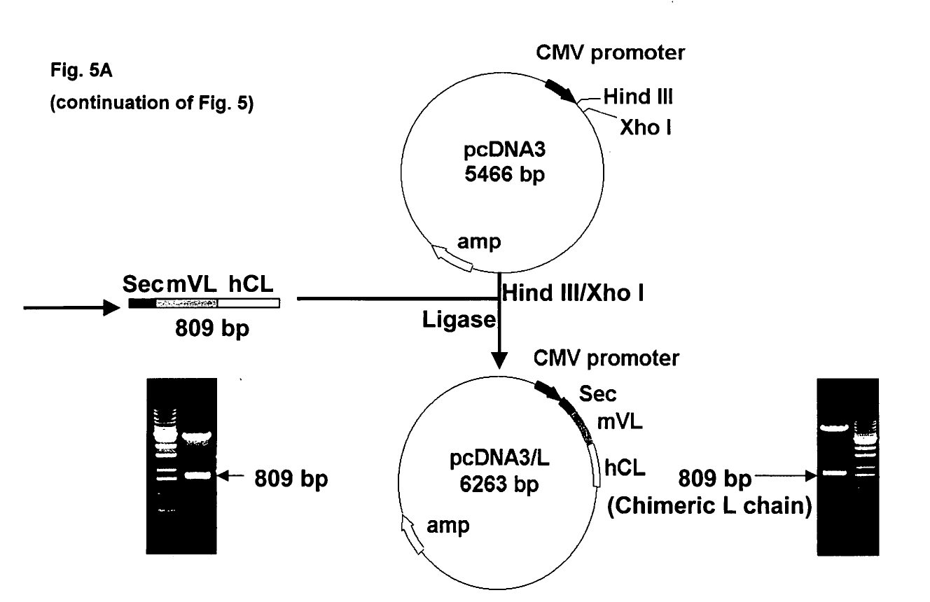

FIGS. 5 and 5A show a scheme for the construction of a chimeric light chain.

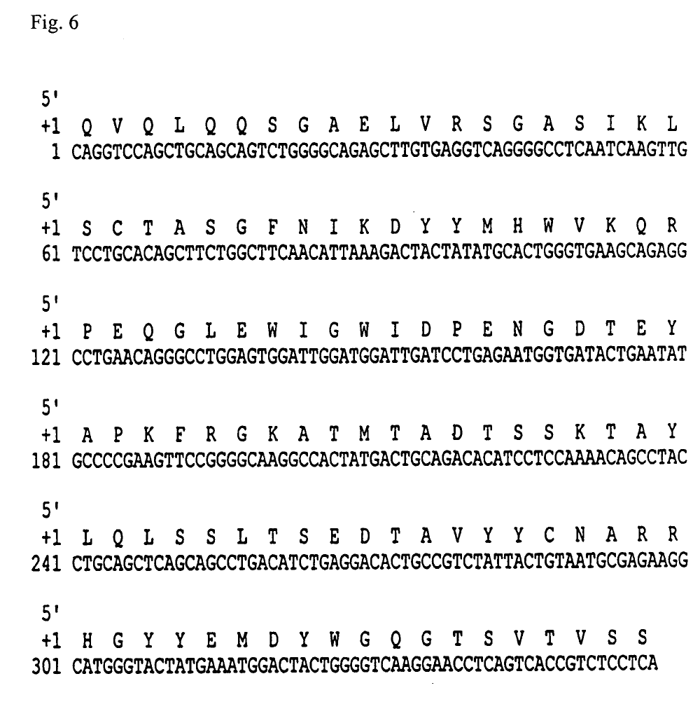

FIG. 6 shows the nucleotide sequence of 2A2 monoclonal antibody heavy chain variable region (in the sequence listing represented as SEQ ID NO: 1). The deduced amino acid region is shown in the top row (in the sequence listing represented as SEQ ID NO: 2).

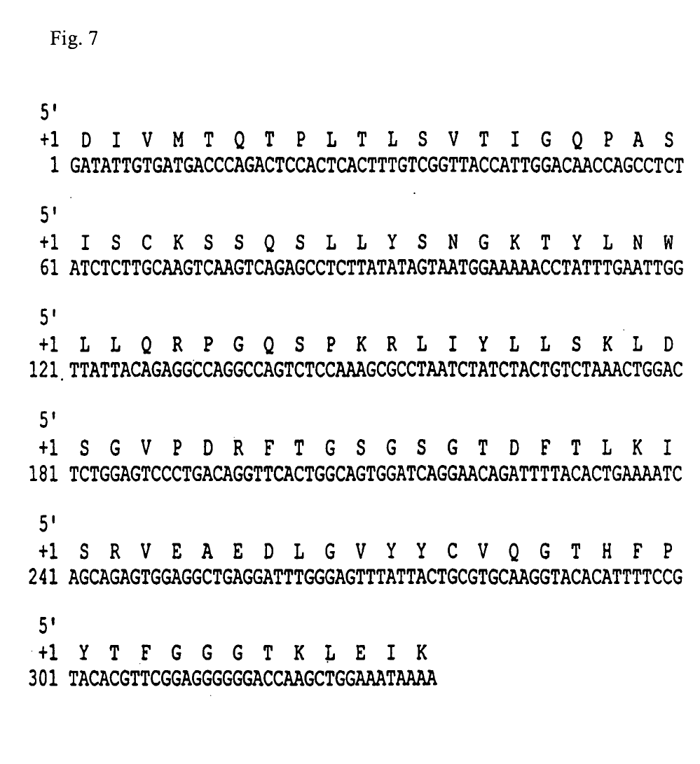

FIG. 7 shows the nucleotide sequence of 2A2 monoclonal antibody light chain variable region (in the sequence listing represented as SEQ ID NO: 3). The deduced amino acid region is shown in the top row (in the sequence listing represented as SEQ ID NO: 4).

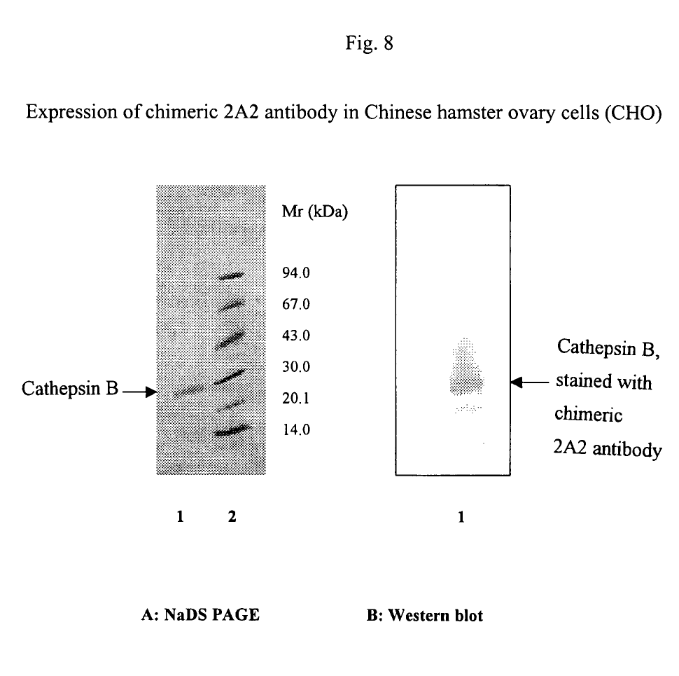

FIG. 8 shows PAGE and Western blot of human cathepsin B, stained with chimeric 2A2 antibody, expressed in Chinese hamster ovary (CHO) cells. A: polyacrylamide gel electrophoresis of recombinant human cathepsin B (1) and of standards (2); B: Western blot—cathepsin B (1) stained with chimeric 2A2 antibody. Goat anti-human antibody (IgG) conjugated with horseradish peroxidase was used as a secondary antibody. As the substrate 0.05% diaminobenzidine (DAB) and 0.01% of H₂O₂ in 0.05M Tris/HCl buffer, pH 7.5 were used.

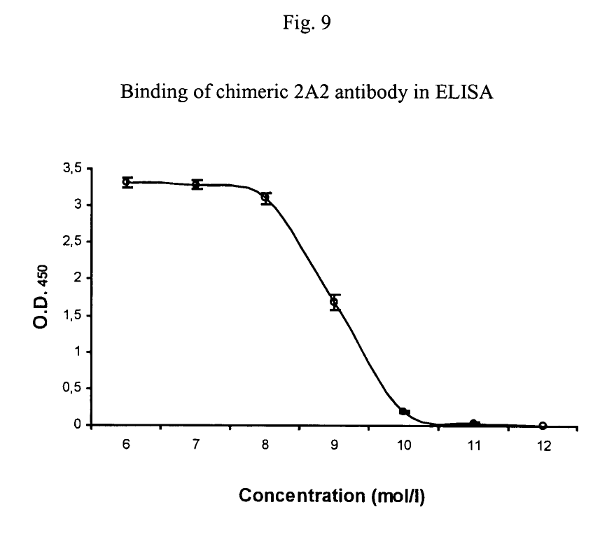

FIG. 9 shows the binding of chimeric 2A2 antibody in ELISA. Aliquots of purified chimeric antibody 2A2 in molar concentrations (10-6-10-12 M) were added to a microtitre plate coated by cathepsin B (2 μg/ml). ELISA was performed as described (Schweiger et al., J. Immunol. Methods 201: 165-172, 1997).

The antibodies described and claimed herein have the ability to neutralise cathepsin B. In the context of this invention the term ‘neutralising’ shall be defined to mean impairing the biological activity. In this respect it has been found that this impairment seems to account for the property of the subject antibodies to essentially stop the progress of metastasis.

The antibodies of the present invention may be prepared in any animal available and suitable for antibody production such as mouse, rabbit or chicken. Yet, when used in humans such antibodies are immunogenic with the effect that the individual to be treated will eventually evoke an immune response against the antibodies administered. For these reasons the antibodies may be redesigned such as by means of chimerisation. To this end the unmodified non-human variable domains are linked with human constant regions of light chain and heavy chains by means of recombinant gene technology and a chimeric antibody is produced in suitable cells. On this way the binding affinity of the original non-human antibody is preserved while the immunogeneicity is significantly reduced.

In a further step the antibody may also be humanised. For achieving this objective the variable regions of the non-human part of the antibody are adapted to human conformations. The techniques for preparing humanised antibodies are well known in the art, e.g. as indicated in Hurle and Gross, Curr. Opin. Biotechnol. 5: 428-433, 1994.

In view of the foregoing, the term ‘antibody’ shall be interpreted to comprise animal antibodies, chimeric antibodies, humanised antibodies, but also mini-antibodies of the mentioned types, preferably fragments, such as Fab, Fv and/or scFv parts.

The antibodies, modified as described above, can be produced in suitable cells such as E. coli, yeasts or mammalian cells. Yet, for conformational and immunogenic reasons mammalian cells are preferred since they may provide a glycosylation pattern resembling that of normal human cells.

According to a preferred embodiment the heavy chain and light chain variable regions of a monoclonal antibody of the present invention are as shown in the Sequence Listing attached hereto and represented as SEQ ID NO: 1, SEQ ID NO: 2, SEQ ID NO: 3 and SEQ ID NO: 4.

A hybridoma cell line capable to express an antibody of the present invention was deposited on 17 May 2001 with the Deutsche Sammlung von Mikroorganismen und Zellkulturen GmbH (DSMZ), Mascheroder Weg 1b, D-38124 Braunschweig, Germany and received the accession No. DSM ACC2506. This hybridoma cell line also represents an object of the present invention.

CHO clone C6A2/CHO capable of stabile production of 2A2 chimeric antibody was deposited on 6 Mar. 2002 at the Deutsche Sammlung von Mikroorganismen und Zellkulturen GmbH (DSMZ), Mascheroder Weg 1b, D-38124 Braunschweig, Germany with the accession number DSM ACC2537. This clone also represents an object of the present invention.

It can be shown that the antibodies of the present invention significantly decrease the invasion of tumour cells through Matrigel, an artificial matrix resembling normal tissue. Therefore the antibodies of the present invention may be used for treating and/or diagnosing diseases associated with an increased concentration and/or activity of cathepsin B such as cancer or arthritis. In particular, cancers such as e.g. breast, brain, colorectal, lung, head and neck, prostate, ovarian, melanoma cancers may be successfully treated with the antibody of the present invention. Also tumour angiogenesis may be inhibited by using the neutralising antibody according to the present invention.

It has surprisingly been found that cathepsin B probably plays a role in the onset and development of arthritis. Consequently, the antibodies of the present invention may also be used in this respect.

The antibodies may be formulated in any galenic form deemed to be appropriate, such as solutions or powders for solutions for parenteral i.e. subcutaneous, intramuscular or intraveneous administration. Any drug delivery systems such as lyposomes, stealth lyposomes, microspheres and solid nanoparticles for intranasal or other interventions may be used. The antibodies may be used in conjunction with any substances such as toxins, radionucleotides, other monoclonal antibodies, chemotherapeutics and immunosuppressive agents which may enhance their targeting and therapeutic effect.

Hence, the present invention also refers to a pharmaceutical composition comprising an antibody as described herein. It will be appreciated that, depending on the route of administration, the pharmaceutical composition will contain carriers or excipients usually utilised. In addition, the attending physician will be expected to choose the appropriate route of administration taking into account the corresponding state of disease to be treated.

The present invention is illustrated by the following nonlimiting examples:

### EXAMPLE

1. Immunisation

In order to prepare specific monoclonal antibodies, mice were immunised by highly purified recombinant human cathepsin B expressed in E. coli (Kuhelj et al., Eur. J. Biochem. 229: 533-539, 1996). Four BALB/c mice were immunised subcutaneously with cathepsin B (25 μg/mouse) emulsified in complete Freund's adjuvant, followed by intraperitoneal injections of the same amount of antigen in incomplete Freund's adjuvant on days 14, 28 and 42. On day 49 test bleeds were taken and the titre of anti-cathepsin B specific antibodies was determined using antigen immobilised ELISA. The mouse with the highest titre was boosted intraperitonally on days 56 and 57 with cathepsin B (30 μg/mouse) in saline solution, and on day 59 used for fusion.

Hybridoma Production

For hybridoma production 9.5×10⁶ splenocytes and 5.6×10⁶ myeloma cells (NS-1/1-Ag4-1) were fused using PEG (Koehler and Milstein, Nature 256: 495-497, 1975). After fusion, hybridoma cells were grown on 96-well cell culture plates using HAT supplemented DMEM medium. After HAT selection the supernatants of hybridoma cells were tested for production of antibodies specific for cathepsin B by using antigen immobilised ELISA.

Screening of Hybridoma Cells Producing Neutralising Anti-Cathepsin B Antibodies

Supernatants of hybridomas positive for production of antibodies against cathepsin B were further tested for inhibitory activity against cathepsin B using fluorimetric assay and synthetic substrate Z-Arg-Arg-AMC (Bachem, Switzerland). The screening was performed on 96-well fluorimetric microtitre plates. Cathepsin B (10 μl, 5×10⁻⁸M), activation buffer (30 μl, 4.5 mM cysteine) and supernatants (50 μl) were preincubated for 30 minutes, then the substrate (10 μl, 5 μM) was added and it was additionally incubated for 15 minutes. The reaction was blocked by adding iodacetate (100 μl, 1 mM). Z-Arg-Arg-AMC was cleaved by cathepsin B into a fluorescent product 7-amino-4-metilcoumarin. Its presence was detected in the fluorimeter using excitation wavelength of 370 nm and emission wavelength of 460 nm. DMEM was used in the control sample. 24 clones exhibiting the highest inhibitory effect were subcloned on 24-well microtitre plates.

After 10 days the supernatants from individual clones were tested to Z-Arg-Arg-AMC at the same conditions as described above. 10 clones with the highest inhibition of cathepsin B activity were transferred first to 25 cm² and subsequently to 75 cm² culture flasks. Antibodies were isolated from supernatants using affinity chromatography on Protein A Sepharose.

Purified antibodies were tested for inhibitory activity against cathepsin B first by using Z-Arg-Arg-AMC as described above and then by using fluorescent BODIPY FL dye-labelled casein (Molecular Probes, USA). For the latter, cathepsin B (20 μl, 1×10⁻⁷M) was first pre-incubated with the activator (10 mM cysteine in MES buffer, pH 6.0) for 15 minutes. Subsequently, monoclonal antibodies (50 μl, 1×10⁻⁷M) and substrate (100 μl, 10 μg/ml) were added and the mixture was incubated for 1 hour on a plate shaker at 20° C., protected from light. The content of released fluorescent BODIPY FL dye-labelled peptides corresponded to the level of active cathepsin B. Fluorescent peptides were detected with excitation/emission wavelengths 485/538 nm. Cathepsin B incubated without antibodies was used as a positive control. The decrease in fluorescence measured in the samples in the presence of antibodies indicated the inhibitory activity of isolated antibodies.

2. Biochemical Characterisation of Selected Inhibitory Antibodies

Inhibition of Tumor Cell Invasion by the Neutralising Antibodies

The human breast epithelial cell line MCF 10A neoT was derived from a parental immortalized cell line MCF10A (Soule et al., Cancer Res. 50: 6075-6086, 1990) by transfection using a plasmid containing a neomycin-resistant gene and human T-24 mutated Ha-ras oncogene (Ochieng et al., Invasion Metastasis 11: 38-47, 1991), and was obtained with Prof. B. Sloane, Wayne State University, Detroit.

The cells were cultured up to 80% confluence as monolayers in 75 cm² plastic cell culture flasks (Falcon, USA) in DMEM/F12 medium (1:1) supplemented with 12.5 mM HEPES (Sigma, USA), 5% foetal bovine serum (Hyclone, USA), 10 μg/ml insulin, 0.5 μg/ml hydrocortisone, 0.02 μg/ml epidermal growth factor (all Sigma, USA) and antibiotics (penicillin, streptomycin, Krka, d.d., Slovenia), at 37° C. and 5% CO₂. For subculturing, the cells were detached by 0.05% trypsin and 0.02% ethylenediaminetetraacetate (EDTA) in phosphate buffered saline (PBS). Prior to their use in invasion and viability assays, 0.4% EDTA and 0.1% bovine serum albumin (BSA) in PBS, pH 7.4 were used for detaching. The viability of the cells used in experiments was at least 90% as determined by staining with nigrosin. The cells were grown in the presence of foetal bovine serum depleted of cysteine proteinase inhibitors by affinity chromatography on a CM papain-Sepharose column (Kos et al., 1992). Briefly, 20 ml of serum diluted 1:2 v/v with 0.02 M PBS buffer, pH 7.4 were incubated with 10 ml CM papain-Sepharose (Pharmacia, Sweden) for 20 minutes and packed in a column. Fractions (3 ml) were tested for residual inhibitory activity with BANA (Bz-DL-Arg-2-Nnap, Serva, Germany) and stored at −20° C. until use.

The cells were quantitated by the MTT colorimetric assay as described (Mosmann, J., Immunol. Methods 65: 55-63, 1983). The assay is based on the cleavage of the yellow tetrazolium salt, 3-4,5 dimethylthiazol-2,5 diphenyl tetrazolium bromide (MTT) (Sigma, USA), into water-insoluble formazan crystals by the mitochondrial enzyme succinate-dehydrogenase present in living cells. The formazan crystals were solubilised using isopropanol and measured for optical density on ELISA reader (SLT, Rainbow) at 570 nm, reference filter 690 nm.

The effect of neutralising monoclonal antibodies was compared to the effects of the following natural and synthetic inhibitors of cysteine proteinases:
1. Irreversible inhibitor E-64, trans-epoxysuccinyl-L-leucylamido-(4-guanidino) butane (Sigma, USA)—general inhibitor of cysteine proteinases (Barret et al., Biochem. J. 201: 189-198, 1982).
2. Reversible tight-binding protein inhibitor chicken cystatin—general inhibitor of cysteine proteinases (Kos et al., Agents Actions 38: 331-339, 1992).
3. CLIK-148,—epoxysuccinyl peptide derivative (Premzl et al., Biol. Chem. 382: 2001) provided by Prof. Nobuhiko Katunuma, Tokushima Bunri University, Japan—inhibitor of cathepsin L.
4. Pepstatin A (Sigma, USA)—inhibitor of cathepsin D.
5. SQAPI-like inhibitor—protein inhibitor of cathepsin D isolated from squash Cucurbita pepo (Christeller et al., Eur. J. Biochem. 254: 160-167, 1998).

The cytotoxicity of the neutralising monoclonal antibodies and inhibitors was tested as described in the literature (Holst-Hansen and Brünner, Cell Biology, A Laboratory Handbook, 2^{nd} ed. (Academic Press), pp. 16-18, 1998). Briefly, cells were added to a final concentration of 5×10⁴ cells/200 μl per well of a 96-well microtitre plate (Costar, USA). Appropriate concentrations of the monoclonal antibody, inhibitor or control medium were added. The plates were incubated for 24 hours at 37° C. and 5% CO₂. The medium was carefully removed, 200 μl of 0.5 mg/ml MTT were added and it was incubated for three hours at 37° C. and 5% CO₂. The medium was removed and formazan crystals were dissolved in 200 μl/well of isopropanol. The absorbance was measured as described above. All tests were performed in quadruplicate.

The effects of the monoclonal antibody and of proteinase inhibitors upon invasion were tested using a modified method as described in the literature (Holst-Hansen et al. Clin. Exp. Metastasis 14: 297-307, 1996). Transwells (Costar, USA) with 12 mm polycarbonate filters, 12 μm pore size, were used. 25 μl of 100 μg/ml fibronectin (Sigma, USA) were applied on the lower side of the filters, which were left for one hour in a sterile chamber to dry. The upper side of the filters was coated with 100 μl of 1 mg/ml Matrigel (Becton Dickinson, USA) and 100 μl of DMEM/F12 were added. The Matrigel was dried overnight at room temperature in a sterile chamber and reconstituted with 200 μl of medium for one hour at 37° C. The upper compartments were filled with 0.5 ml of the cell suspension, final concentration 4×10⁵ cells/ml, containing the appropriate concentration of the inhibitor. The lower compartments were filled with 1.5 ml of the medium containing the same concentration of the inhibitor. The plates were incubated for 24 hours at 37° C. and 5% CO₂. MTT was added to a final concentration of 0.5 mg/ml to the upper and lower compartments and the plates were incubated for additional 3 hours. Media from either compartment were separately transferred to Eppendorf tubes and centrifuged at 6200 rpm for 5 minutes. The supernatants were discarded and the remaining formazan crystals were dissolved in 1 ml of isopropanol. The colour intensity was measured as described above. As controls, the cells were incubated with a medium containing the appropriate volumes of methanol, distilled water and 50 mM NaHCO₃, 0.3 M NaCl, pH 7.5, the solvents used for the preparation of concentrated solutions of the monoclonal antibody and inhibitors. The invasion was recorded as the percentage of cells that penetrated the Matrigel-coated filters in comparison to controls and was calculated as OD_{lower}/OD_{lower}+ODᵤₚₚₑᵣ×100. All tests were performed in triplicate.

3. Construction and Expression of Chimeric Antibody

Preparation of the Total RNA from Hybridoma Producing the Monoclonal Antibody (MAb) Against Cathepsin B

The total RNA was isolated from the 1.58×10⁸ 2A2 hybridoma cell line by using the guanidinium method.

Synthesis of the First Strand of cDNA

The cDNAs were synthesised by RT-PCR.

Amplification of Genes of V_{L} and V_{H} of MAb by PCR and Determination of Their Sequences

Two pairs of primers were used for the PCR:

For the light chain:

| | |
|---|---|
| A | |
| Forward primer: | |
| NK4: 5′-GATGGATATCGTGCTGACCCAATCTC | (SEQ ID NO:5) |
| CAGCTTCTTTGG-3′ | |
| Backward primer: | |
| NK3: 5′-GTGCCTCGAGTCGACTTAGCACTCAT | (SEQ ID NO:6) |
| TCCTGTTGAATCTT-3′ | |
| B | |
| Forward primer: | |
| L5V: 5′-GTGTGCACTCTGATATTGTGATG-3′ | (SEQ ID NO:7) |
| Backward primer: | |
| L3V: 5′-GGTGCAGCCACAGTCCGT | (SEQ ID NO:8) |
| TTTATTTC-3′ | |
| For the heavy chain: | |
| A | |
| Forward primer: | |
| NK-HD5: 5′-GTGAGAGCTCSAGGTSMARCTGC | (SEQ ID NO:9) |
| AGSAGTCT-3′ | |
| Backward primer: | |
| nH3V: 5′-GGTGGTCGACGCTGA | (SEQ ID NO:10) |
| GGAGACGGT-3′ | |
| B | |
| Forward primer: | |
| H5V: 5′-GTGTGCACTCTGAGGTGCAGCTG-3′ | (SEQ ID NO:11) |
| Backward primer: | |
| H3V: 5′-TGGTCGACGCTGAGGAGACGGT-3′ | (SEQ ID NO:12) |

PCR was performed in a GeneAmp PCR System 2400 (PERKIN ELMER) with the light chain (primer NK4 and NK3) and heavy chain primers (primer NK-HD5 and nH3V) within 30 cycles, respectively, at the following conditions: pre-denaturation at 95° C. for 5 minutes; denaturation at 95° C. for 30 seconds; annealing at 50° C. for 30 seconds and extension at 72° C. for one minute. The PCR products were checked on 1% agarose gel and excised for further purification with GENELEAN Kit.

The PCR products for light chain and for heavy chain were cloned into a pUC 19 and pGEM-T Easy vector, respectively. Their sequences were determined with the apparatus ABI PPISM 310 Genetic Analyzer (PERKIN ELMER).

Construction of Chimeric Light and Heavy Chains

A chimeric light chain and a chimeric heavy chain were constructed, respectively. The mouse V_{L} and V_{H} were joined to human IgG constant region (Cκ and C_{H1}, respectively) and were subsequently inserted into an expression vector pcDNA3.

Light Chain

After amplification of V_{L} fragment with primer L5V and L3V at the following conditions: pre-denaturation at 95° C. for 5 minutes; denaturation at 95° C. for 30 seconds; annealing at 55° C. for 30 seconds and extension at 72° C. for one minute, the PCR product was subcloned in pUC/hCK, which contained human Cκ gene. The chimeric light chain was first subcloned into pUTSEC vector which was designed to provide the recombinant chimeric chains with the leader peptide required for the secretion of proteins into the extracellular medium (Li, E. et al., 1997). Finally, the chimeric light chain and the 163 bp genomic sequence encoding mouse heavy chain immunoglobulin secretion signal were cloned into the eukaryotic expression vector pcDNA3. The sequencing was done in each vector to confirm the correct insert.

Heavy Chain

After amplification of V_{H} fragment with primer H5V and H3V at the following conditions: pre-denaturation at 95° C. for 5 minutes; denaturation at 95° C. for 30 seconds; annealing at 55° C. for 30 seconds and extension at 72° C. for one minute, the V_{H} domain PCR product was subcloned into pUTSEC vector and then subcloned into pUC/hIgG1 vector containing the gene for the human Cγ1 region. Also, the chimeric heavy chain was cloned into the eukaryotic expression vector pcDNA3. The sequencing was done in each vector to confirm the correct insert sequence.

Transfection of Recombinant Light Chain and Heavy Chain into Sp 2/0 Murine Myeloma Cells or Chinese Hamster Ovary (CHO) Cells.

Approximately 1×10⁷ mouse myeloma Sp2/0 cells or CHO cells were resuspended in 0.5 ml of cold PBS (10.1 mM Na₂HPO₄, 1.8 mM KH₂PO₄, 137 mM NaCl, 3 mM KCl, pH 7.2) and put in a cuvette for electroporation with an electrode gap of 0.4 cm; 10 μg of Bgl II-linearized plasmids (light chain -pcDNA3 and heavy chain -pcDNA3 purifying plasmids) were added to the cells and electroporation was performed with a single pulse at 960 μF, 290 V, in a Bio-Rad Gene Pulser equipped with a capacitance extender. After electroporation, the cells were kept on ice for 5-10 minutes, washed, resuspended in 30 ml od 10% FCS RPMI 1640 medium and seeded in 10 cm dishes at a density of 3-4×10⁵ cells/dish.

After 24 hours a selective medium containing G-418 at a final concentration of 400 μg/ml was added. The supernatants of the selected clones were screened by ELISA on plates coated with cathepsin B to detect the presence of secreted chimeric MAb. Western blots were also used to check the expression product and affinity of chimeric MAbs.

The chimeric antibody was isolated and tested for inhibition of tumour cell invasion as described above for murine antibodies.

Sequences

The following sequences are contained within this application:
SEQ ID NO: 1: nucleotide sequence of 2A2 monoclonal antibody heavy chain variable region
SEQ ID NO: 2: amino acid region deduced from nucleotide sequence of 2A2 monoclonal antibody heavy chain variable region
SEQ ID NO: 3: nucleotide sequence of 2A2 monoclonal antibody light chain variable region
SEQ ID NO: 4: amino acid region deduced from nucleotide sequence of 2A2 monoclonal antibody light chain variable region
SEQ ID NO: 5: forward primer for light chain—NK4
SEQ ID NO: 6: backward primer for light chain—NK3
SEQ ID NO: 7: forward primer for light chain—L5V
SEQ ID NO: 8: backward primer for light chain—L3V
SEQ ID NO: 9: forward primer for heavy chain—NK-HD5
SEQ ID NO: 10: backward primer for heavy chain—nH3V
SEQ ID NO: 11: forward primer for heavy chain—H5V
SEQ ID NO: 12: backward primer for heavy chain—H3V
   Sequence Listing Free Text

The following free text is contained in the Sequence Listing:
SEQ ID NO: 9: Description of Artificial Sequence: forward primer for heavy chain with additional restriction sites

1. A monoclonal antibody directed against cathepsin B neutralising its enzymatic activity.

2. The antibody according to claim 1, wherein the antibody comprises murine variable regions and human constant regions (chimeric antibody).

3. The antibody according to claim 2, wherein monoclonal antibody heavy chain and light chain variable regions are as represented by SEQ ID NO: 2, SEQ ID NO: 2, SEQ ID NO: 3 and SEQ ID NO: 4.

4. The antibody according to claim 1, wherein the antibody is humanised.

5. The antibody according to claim 1, wherein the antibody is a mini-antibody.

6. A cell expressing the monoclonal antibody according to claim 1.

7. The cell according to claim 6, which was deposited on 17 May 2001 with Deutsche Sammlung von Mikroorganismen und Zellkulturen GmbH (DSMZ), under the accession No. DSM ACC2506.

8. A clone capable of stable production of the chimeric antibody according to claim 1, which was deposited on 06.03.2002 with Deutsche Sammlung von Mikroorganismen und Zellkulturen GmbH (DSMZ), under the accession No. DSM ACC2537.

9. A use of the antibody according to claim 1 for the treatment and/or diagnosing of a disease associated with an increased cathepsin B activity.

10. A use according to claim 9, wherein the increased activity is derived from an increased concentration of cathepsin B.

11. A use according to claim 9, wherein the disease is cancer or arthritis.

12. A pharmaceutical composition containing an antibody according to claim 1.

13. The antibody according to claim 1 for use in the treatment and/or diagnosing of a disease associated with an increased cathepsin B activity.

14. The antibody according to claim 13, wherein the activity derives from an increased concentration of cathepsin B.

15. The antibody according to claim 13, wherein the disease is cancer or arthritis.

16. Use of the antibody according to claim 1 for manufacturing a medicament for the treatment and/or diagnosing of a disease associated with an increased cathepsin B activity.

17. Use according to claim 16, wherein the increased activity derives from an increased concentration of cathepsin B.

18. Use according to claim 16, wherein the disease is cancer or arthritis.

19. The antibody according to claim 2, wherein the antibody is humanised.

20. The antibody according to claim 3, wherein the antibody is humanised.


SEQUENCE LISTING (12 sequences)

SEQ ID NO: 1 - DNA, 357 bp, Mouse
caggtccagctgcagcagtctggggcagagcttgtgaggtcaggggcctcaatcaagttgtcctgcacagcttctggcttcaacattaaagactactatatgcactgggtgaagcagaggcctgaacagggcctggagtggattggatggattgatcctgagaatggtgatactgaatatgccccgaagttccggggcaaggccactatgactgcagacacatcctccaaaacagcctacctgcagctcagcagcctgacatctgaggacactgccgtctattactgtaatgcgagaaggcatgggtactatgaaatggactactggggtcaaggaacctcagtcaccgtctcctca

SEQ ID NO: 2 - protein, 119 aa, Mouse
QVQLQQSGAELVRSGASIKLSCTASGFNIKDYYMHWVKQRPEQGLEWIGWIDPENGDTEYAPKFRGKATMTADTSSKTAYLQLSSLTSEDTAVYYCNARRHGYYEMDYWGQGTSVTVSS

SEQ ID NO: 3 - DNA, 336 bp, Mouse
gatattgtgatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctactgtctaaactggactctggagtccctgacaggttcactggcagtggatcaggaacagattttacactgaaaatcagcagagtggaggctgaggatttgggagtttattactgcgtgcaaggtacacattttccgtacacgttcggaggggggaccaagctggaaataaaa

SEQ ID NO: 4 - protein, 112 aa, Mouse
DIVMTQTPLTLSVTIGQPASISCKSSQSLLYSNGKTYLNWLLQRPGQSPKRLIYLLSKLDSGVPDRFTGSGSGTDFTLKISRVEAEDLGVYYCVQGTHFPYTFGGGTKLEIK

SEQ ID NO: 5 - DNA, 38 bp, Mouse
gatggatatcgtgctgacccaatctccagcttctttgg

SEQ ID NO: 6 - DNA, 40 bp, Mouse
gtgcctcgagtcgacttagcactcattcctgttgaatctt

SEQ ID NO: 7 - DNA, 23 bp, Mouse
gtgtgcactctgatattgtgatg

SEQ ID NO: 8 - DNA, 26 bp, Mouse
ggtgcagccacagtccgttttatttc

SEQ ID NO: 9 - DNA, 31 bp, Artificial Sequence
  Description of Artificial Sequenceforward primer for heavy chain with additional restriction sites
gtgagagctcsaggtsmarctgcagsagtct

SEQ ID NO: 10 - DNA, 24 bp, Mouse
ggtggtcgacgctgaggagacggt

SEQ ID NO: 11 - DNA, 23 bp, Mouse
gtgtgcactctgaggtgcagctg

SEQ ID NO: 12 - DNA, 22 bp, Mouse
tggtcgacgctgaggagacggt